(19) United States
(12) Patent Application Publication
(10) Pub. No.: US 2016/0114077 A1
(43) Pub. Date: Apr. 28, 2016

(54) ELASTOMERIC AND DEGRADABLE POLYMER SCAFFOLDS AND HIGH-MINERAL CONTENT POLYMER COMPOSITES, AND IN VIVO APPLICATIONS THEREOF

(71) Applicant: UNIVERSITY OF MASSACHUSETTS MEDICAL SCHOOL, Boston, MA (US)

(72) Inventors: Jie Song, Shrewsbury, MA (US); Artem Kutikov, Worcester, MA (US)

(21) Appl. No.: 14/894,360

(22) PCT Filed: May 30, 2014
(86) PCT No.: PCT/US14/40194

§ 371 Date: Nov. 26, 2015

Related U.S. Application Data
(60) Provisional application No. 61/829,671, filed on May 31, 2013.

(51) Int. Cl. A61L 27/18; A61L 27/58; A61L 27/46; A61F 2/28; C08G 63/91

(52) U.S. Cl. CPC A61L 27/18; A61F 2/2846; C08G 63/912; A61L 27/46; A61L 27/58; A61F 2002/2835; A61F 2002/2817; A61F 2210/0004; A61F 2210/0014; A61F 2210/0061; A61F 2210/0085; A61F 2310/0097

(57) ABSTRACT

This invention provides novel synthetic bone grafting materials or tissue engineering scaffolds with desired structural and biological properties (e.g., well-controlled macroporosities, spatially defined biological microenvironment, good handling characteristics, self-anchoring capabilities and shape memory properties) and methods of their applications in vivo.

### PRIORITY CLAIMS AND CROSS REFERENCE TO RELATED APPLICATIONS

This application claims the benefit of priority from U.S. Provisional Application Ser. No. 61/829,671, filed May 31, 2013, the entire content of which is incorporated herein by reference in its entirety.

### GOVERNMENT RIGHTS

The United States Government has certain rights to the invention pursuant to Grant Nos. R01AR055615 and R01GM088678 awarded by the National Institutes of Health and Grant No. W81XWH-10-0574 awarded by the Department of Defense to the University of Massachusetts.

### TECHNICAL FIELDS OF THE INVENTION

The invention generally relates to polymer compositions. More particularly, the invention relates to polymer scaffolds and composites of biodegradable amphiphilic polymers and inorganic minerals as well as methods for their preparation and uses thereof, for example, in bone grafting and tissue engineering applications.

### BACKGROUND OF THE INVENTION

Bone tissue engineering approaches aim to overcome the limitations of autografts and allografts for the repair of critical-size bone defects, which include donor site morbidity, limited quantity, high failure rate or risk for infections. (Faour, et al. 2011 Injury 42 Suppl 2, S87-90.) Tissue engineering typically employs degradable biomaterial scaffolds to support the delivery of cells or therapeutics to the defect site to guide and promote tissue regeneration and eventually be replaced by the regenerated tissue of interest. (Langer, et al. 1993 Science 260, 920-926.)

Significant research effort has been devoted to the development of degradable polymer or bioceramic composite materials for musculoskeletal tissue engineering. Such materials combine synthetic polymers with biominerals such as hydroxyapatite (HA), the principle mineral component of bone. HA provides the necessary mechanical strength, enhances the material's osteoconductivity, serves an important source for calcium and phosphate ions, and plays an important role in retaining proteins and support bone cell attachment and growth factor binding and release. Characterized with its high stiffness and brittleness, however, HA alone is not well suited for broad orthopedic applications beyond serving as a non-weight bearing bone void filler. To address such limitations, HA has been incorporated with synthetic polymers to form 2- or 3-dimensional, dense or porous composite scaffolds. Adequate interfacial adhesion/affinity between HA and the polymeric component is a key to achieving structural and mechanical integrity.

Biodegradable amphiphilic block co-polymers, such as those composed of poly(D,L-lactic acid)-poly(ethylene glycol)-poly(D,L-lactic acid) (PELA) are promising materials for medical implant applications. (WO 2013/044078 A2 by Song, et al. and references cited therein.) These polymers can be blended with high percentages of HA and electrospun into membranes or rapid prototyped into 3-dimensional (3-D) scaffolds. Such materials are highly elastic, hydrophilic, and exhibit improved bioactivity when compared to poly(D,L-lactic acid)-HA composites. While electrospun HA-PELA would find unique orthopedic applications (e.g., as synthetic periosteal membrane wrapped around structural allografts), their limited thickness and porosity make them less suited for treating large defects where sufficient nutrient transport and cellular ingrowth throughout a 3-D macroporous scaffold is desired.

Stable implant fixation, particularly in bone and bone/soft tissue interface and osteochondral applications, is critical for the ultimate performance of the implant. One approach is swelling induced self-fixation or anchoring, which takes advantage of the swelling characteristics of polymers.

U.S. Pat. No. 5,824,079 discloses a crosslinked co-polymer system composed of methyl methacrylate and acrylic acid reinforced with carbon and Kevlar fibers. This is designed to be used as a swelling bone/soft-tissue anchor. This bone anchor is non-biodegradable and not osteoconductive. Thus, it is unsuitable as a bone graft or tissue engineering scaffold. Furthermore, the thermoset polymer composition makes the material unsuitable for extrusion, rapid prototyping, and other techniques commonly used to fabricate bone grafts and tissue engineering scaffolds.

U.S. Pat. No. 5,084,050 discloses a hollow cylindrical bone anchor designed to stabilize bone screws or implants by swelling. This swellable anchor can be composed of degradable polymers from the polylactate group and inorganic minerals such as apatite. The anchor was not designed as a bone grafting material or tissue engineering scaffold. Additionally, the materials weaken upon hydration, limiting their ultimate anchoring potential.

Rapid prototyping techniques such as selective laser sintering (SLS), powder-based three-dimensional printing (3DP), and fused deposition modeling (FDM) have been employed in the fabrication of large 3-D scaffolds for bone tissue engineering. (Leong, et al. 2003 Biomaterials 24, 2363-2378; Butscher, et al. 2011 Acta Biomater. 7, 907-20.) These rapid prototyping approaches have been used for the formation of HA or HA/polymer composite scaffolds with defined geometries and controlled interconnected pore architecture. (Sun, et al. 2012 J. Biomed. Mater. Res. A 100, 2739-49; Moroni, et al. 2006 Biomaterials 27, 974-85.)

While rapid prototyping technology has become increasingly refined, the selection of biomaterials suitable for prototyping has remained limited. The most widely prototyped polymers are hydrophobic polyesters such as poly(ε-caprolactone) (PCL), poly(L-lactic acid) (PLLA), or poly(lactic acid-co-glycol acid) (PLGA). (Hor, et al. 2007 Biomaterials 28, 5291-7; Williams, et al. 2005 Biomaterials 26, 4817-27; Wiria, et al. 2007 Acta Biomater. 3, 1-12; Schantz, et al. 2005 J. Mater. Sci. Mater. Med. 16, 807-19; Heo, et al. 2009 Tissue Eng. Part A 15, 977-89; Giordano, et al. 1996 J. Biomater. Sci. Polym. Ed. 8, 63-75; Kim, et al. 2012 Biofabrication 4, 025003.) Rapid prototyped amphiphilic polymer scaffolds composed of polyethyleneoxide-terephthalate (PEOT) and polybutylene-terephthelate (PBT) (PEOT/PBT) using a specialized printer have been explored for cartilage tissue engineering. (Woodfield, et al. 2004 Biomaterials 25, 4149-61; Moroni, et al. 2005 J. Biomed. Mater. Res. A 75, 957-65; Leferink, et al. 2013 J. Tissue Eng. Regen. Med. doi:10.1002/term.1842.) However, the PBT component is not biodegradable, resulting in crystalline, hard-to-resorb remnants upon degradations in vivo. (Deschamps, et al. 2004 Biomaterials 25, 247-258.) Furthermore, rapid prototyping HA-PEOT/PBT composite scaffolds for bone tissue engineering was not explored.

Thus, an un-met need continues to exist for novel synthetic tissue scaffolds with desired structural and biological properties (e.g., well-controlled macroporosities, spatially defined biological microenvironment, and good handling characteristics) suitable for use in a wide variety of in vivo applications.

### SUMMARY OF THE INVENTION

This invention provides novel synthetic bone grafting materials or tissue engineering scaffolds with desired structural and biological properties (e.g., well-controlled macroporosities, spatially defined biological microenvironment, good handling characteristics, self-anchoring capabilities and shape memory properties) and methods of their applications in vivo.

For example, disclosed herein is the rapid prototyping of PELA or HA-PELA into 3-D macroporous tissue engineering scaffolds. These are a class of amphiphilic degradable biomaterials shown to exhibit exciting physical (e.g., hydrophilicity, mechanical integrity, and hydration-induced structural rearrangement and mechanical strengthening effect) and biological properties (e.g., osteoconductivity & up-regulated osteogenic gene expression). An unmodified, consumer-grade 3-D printer can be used for the scaffold fabrication, facilitating the translation of this promising biomaterial to tissue engineering applications and promoting its wider use by the research community. The rapid prototyped PELA and HA-PELA composite scaffolds demonstrated unique swelling and mechanical properties that translated into hydration-induced self-fixation behavior.

The self-fixation behavior is uniquely suited for scaffold-assisted tissue engineering applications where the ability of a scaffold to conform and secure itself within a tissue defect is desired for its stable implantation. Also demonstrated were differential abilities of rapid prototyped PELA and HA-PELA scaffolds to suppress or support the adhesion and proliferation of NIH3T3 fibroblasts and MSCs. These cell-adhesion properties can potentially be exploited in biphasic constructs with spatially controlled cell adhesion.

The self-anchoring capabilities result from a combination of swelling and hydration-induced stiffening of the polymer network. Additionally, the superior thermoplastic properties of the composite materials disclosed herein allow them to be rapid prototyped into tissue engineering scaffolds. Thus, the invention represents a significant advance over prior art bone-anchoring materials or fixation devices, which weakened upon hydration, are non-degradable and non-osteoconductive and thus are unsuitable for bone grafting and tissue engineering applications.

For example, the disclosure herein includes the excellent blending of biodegradable, amphiphilic PLA-PEG-PLA (PELA) triblock co-polymer with HA and the fabrication of high-quality rapid prototyped 3-D macroporous composite scaffolds using an unmodified consumer-grade 3-D printer. The rapid prototyped HA-PELA composite scaffolds and the PELA control (without HA) swelled (66% and 44% volume increases, respectively) and stiffened (1.38-fold and 4-fold increases in compressive modulus, respectively) in water. Self-fixation properties of the scaffolds within a confined defect are established and quantified. For example, the peak fixation force measured for the PELA and HA-PELA scaffolds increased 6-fold and 15-fold upon hydration, respectively.

Additionally, the low-fouling 3-D PELA inhibited the attachment of NIH3T3 fibroblasts or MSCs while the HA-PELA readily supported cellular attachment. Furthermore, the feasibility of rapid prototyping biphasic PELA/HA-PELA scaffolds is demonstrated for guided bone regeneration where an osteoconductive scaffold interior encouraging osteointegration and a non-adhesive surface discouraging fibrous tissue encapsulation is desired.

In one aspect, the invention generally relates to a composition comprising a biodegradable amphiphilic block co-polymer. The block co-polymer comprises hydrophilic blocks and degradable hydrophobic blocks and the composition exhibits a shape memory property.

In another aspect, the invention generally relates to an article of manufacture made from a composition disclosed herein.

In yet another aspect, the invention generally relates to a biodegradable medical implant. The implant includes a composition comprising a biodegradable amphiphilic block co-polymer, wherein the block co-polymer comprises hydrophilic blocks and degradable hydrophobic blocks, wherein the implant swells and stiffens upon hydration at body temperature.

In yet another aspect, the invention generally relates to a biodegradable, three-dimensional composite scaffold, prepared by rapid prototyping from a suspension of hydroxyapatite with an amphiphilic block poly(ethylene glycol-co-lactic acid), wherein the composite scaffold exhibits a shape memory property and swells and stiffens upon hydration at body temperature.

In yet another aspect, the invention generally relates to a method for treating a subject in need of bone or tissue grafting or repair. The method includes: providing a biodegradable medical implant comprising a biodegradable amphiphilic block co-polymer comprising a block co-polymer of hydrophilic blocks and degradable hydrophobic blocks, wherein the implant has attached thereto cells or biological agents; and implanting the biodegradable medical implant in a subject in need thereof to assist bone or tissue grafting or repair.

### BRIEF DESCRIPTION OF THE FIGURES

FIG. 1. An illustrative schematic of the fabrication process for 3-D PELA, HA-PELA, and PELA/HA-PELA scaffolds.

FIG. 2. (A) Top, isometric, and side views of scaffold CAD design. (B) Stereomicroscopy images of rapid prototyped HA-PELA and PELA scaffolds. Scale bars=5 mm (C) Scanning electron micrographs of the HA-PELA scaffold. Scale bars=1 mm. (D) Scanning electron micrographs of the PELA scaffold. Scale bars=1 mm.

FIG. 3. Swelling behavior of HA-PELA and PELA scaffolds (n=3). (A) Change in scaffold mass (swelled mass (M)/initial mass (M₀)) over time in deionized water at 37° C. (B) Change in scaffold volume (swelled volume (V)/initial volume (V₀)) over time in deionized water at 37° C. (C) Change in line width after 24 h hydration in deionized water at 37° C. *P<0.05.

FIG. 4. Compressive moduli of dry and hydrated PELA and HA-PELA scaffolds (n=3) at 37° C. *P<0.05.

FIG. 5. Hydration-induced self-fixation test. (A) CAD image of the self-fixation testing device with aluminum spacer. (B) Image of the rapid prototyped self-fixation testing device with HA-PELA scaffold inserted. (C) Image of the testing device secured to the grips of the MTS mechanical testing system. (D) Peak forces required to pull HA-PELA and PELA scaffolds (n=4) out of the self-fixation device before and after hydration. *P<0.05.

FIG. 6. CCK-8 cell viability assay of NIH3T3 attachment and proliferation on HA-PELA and PELA scaffolds (n=3). *P<0.05. (B) Stereomicroscopy images of MTT stained scaffolds 24 h post NIH3T3 seeding. Dark purple stains denote viable cells adhered on the scaffolds. Scale bars=1 mm. (C) CCK-8 cell viability assay of rMSC attached to HA-PELA and PELA scaffolds at 24 h after initial seeding (n=3). *P<0.05.

FIG. 7. PELA/HA-PELA biphasic scaffold. (A) CAD model of the PELA/HA-PELA biphasic scaffold. (B) Stereomicroscopy images of a 6-mm core punched out from the biphasic scaffold. Scale markings=1.0 mm (C) Scanning electron micrograph of a biphasic scaffold (top). Elemental mapping overlay (bottom) of calcium (green) and phosphate (red). Scale bars=1 mm

FIG. 8. Temperature sweep of HA-PELA composites. 0.02% strain amplitude, 1 Hz, 2° C./min.

FIG. 9. Shape memory behavior of rapid prototyped macroporous HA-PELA composites.

FIG. 10. Reprogramming the permanent shape of HA-PELA. (A) Recovery of a permanent flat bar shape from a temporary spiral. (B) Reprogramming into a permanent spiral shape, deformation into flat bar, and subsequent recovery into spiral.

FIG. 11. Illustration of cell-seeded HA-PELA preparation and in vivo implantation.

FIG. 12. Alexa flour 555 phalloidin labeled formalin-fixed rMSCs 24 h after being seeded on 10% HA-PELA (left) or 25% HA-PELA (right) fibrous scaffolds.

FIG. 13. In vivo microCT 3D reconstruction (A) and density-gradient maps of center axial slices (B) of 4- and 12-week of rat femoral segmental defects filled with 10% HA-PELA scaffold pre-seeded with 100,000 rMSCs/cm². The microCT threshold applied precludes the visualization of the HA-PELA scaffold; but the concentric/spiral new bone formation illustrated by the color map of axial center slices supports the templating effect of the rolled up HA-PELA scaffolds.

FIG. 14. Effects of exogenous rMSC seeding density applied to the 10% HA-PELA scaffold on the efficacy of templated bone healing. microCT scans were taken 12 weeks after implantation. Some bone formation was evident even without pre-seeded rMSCs, supporting the osteoconductivity of the HA-PELA scaffold enabling the recruitment of endogenous MSCs. Increasing exogenous rMSC seeding density resulted in greater bone formation.

FIG. 15. In vitro release of rhBMP-2 from HA-PELA scaffolds with varying HA weight percentages in PBS at 37° C. as quantified by ELISA (R&D Systems). The electrospun HA-PELA scaffolds enabled controlled release of rhBMP-2 loaded on the scaffold (235 ng rhBMP2/cm², 75 ng/scaffold). The retention of rhBMP-2 increases with HA content of the HA-PELA scaffold.

FIG. 16. Alkaline phosphatase staining for C2C12 cells cultured on HA-PELA scaffold with (left) and without (right, negative control) preloaded rhBMP-2 (initial loading: 235 ng/cm² rhBMP-2; scaffold incubated in PBS for 1 week prior to being retrieved for cell seeding). The bioactivity of retained rhBMP-2 after 1-week incubation in PBS was confirmed by the ability to induce osteogenic trans-differentiation of C2C12 cells seeded onto the 10% HA-PELA as evidenced by the positive (red) stains for osteogenic marker ALP after 3-day culture (left).

FIG. 17. In vivo microCT 3D reconstruction of a 4-week post-op rat femoral segmental defect filled with 10% HA-PELA scaffold pre-loaded with 500-ng rhBMP-2. Prior to implantation, the scaffold was rolled into a cylinder; rhBMP-2 (10 ng/μL in PBS) was applied and allowed to adhere for 15 min.

### DESCRIPTION OF THE INVENTION

This invention provides novel synthetic bone grafting materials or tissue engineering scaffolds with desired structural and biological properties (e.g., well-controlled macroporosities, spatially defined biological microenvironment, good handling characteristics, self-anchoring capabilities and shape memory properties) and methods of their applications in vivo. The self-anchoring capabilities result from a combination of swelling and hydration-induced stiffening of the polymer network. Additionally, the superior thermoplastic properties of the composite materials disclosed herein allow them to be rapid prototyped into tissue engineering scaffolds. The invention represents significant advance over prior art bone-anchoring materials or fixation devices, which weaken upon hydration, are non-degradable and non-osteoconductive and are unsuitable for bone grafting and tissue engineering applications.

Two major factors hampering the broad use of rapid prototyped biomaterials for tissue engineering applications are the requirement for custom-designed or expensive research-grade 3-D printers and the limited selection of suitable thermoplastic biomaterials exhibiting physical characteristics desired for facile surgical handling and biological properties encouraging tissue integration.

The composite materials and scaffolds of the invention overcome limitations of prior art by using a biodegradable amphiphilic polymer (e.g., PELA) with or without added inorganic mineral (e.g., HA) as self-anchoring bone grafting materials or tissue engineering scaffolds. The thermoplastic biodegradable amphiphilic polymers disclosed herein exhibit hydration-dependent hydrophilicity changes and stiffening behavior, which facilitate the surgical delivery/self-fixation of the scaffold within a physiological tissue environment. Compared to conventional hydrophobic polyesters, these biodegradable amphiphilic polymers present significant advantages in blending with hydrophilic osteoconductive minerals with improved interfacial adhesion for bone tissue engineering applications. For example, PELA or HA-PELA allows the fabrication of custom grafts with tailored macroporosity by rapid prototyping and exhibits an increased modulus upon hydration. This could improve the standard of care by limiting the use of non-degradable fixation devices for bone grafts, enabling ease of bone graft implantation and fixation, and improving the anchoring of osteochondral plugs.

By way of examples, it has been demonstrated the excellent blending of biodegradable, amphiphilic PELA triblock co-polymer with HA and the fabrication of high-quality rapid prototyped 3-D macroporous composite scaffolds using an unmodified consumer-grade 3-D printer. The rapid prototyped HA-PELA composite scaffolds and the PELA control (without HA) swelled (66% and 44% volume increases, respectively) and stiffened (1.38-fold and 4-fold increases in compressive modulus, respectively) in water. Hydration-induced physical changes has been shown to give rise to self-fixation properties of the scaffolds within a confined defect with the peak fixation force measured for the PELA and HA-PELA scaffolds increasing 6-fold and 15-fold upon hydration, respectively. Furthermore, the low-fouling 3-D PELA inhibited the attachment of NIH3T3 fibroblasts or MSCs while the HA-PELA readily supported cellular attachment. Furthermore, rapid prototyping using biphasic PELA/HA-PELA scaffolds was shown to be suitable for guided bone regeneration where an osteoconductive scaffold interior encouraging osteointegration and a non-adhesive surface discouraging fibrous tissue encapsulation is desired.

An exemplary biodegradable amphiphilic block copolymer/hydroxyapatite composite, employed herein to demonstrate the unique capabilities of the invention, is based on poly(ethylene glycol-co-lactic acid). The block co-polymer comprised of hydrophilic and degradable hydrophobic blocks for stable interfacing with HA, resulting in stable polymer-HA suspensions. The hydrophilic blocks are important for HA binding while the hydrophobic blocks allow for degradability and aqueous stability as well as eletrospinability. Thus, the block co-polymer platform fulfills key requirements, including HA integration, ease of processing (such as electrospinnability), aqueous stability, and biodegradability. The length of the PLA and PEG segments can be varied to modify the properties (mechanical, hydrophobicity, degradability) of the final polymer. PLA-PEG-PLA or PEG-PLA-PEG block copolymers can be synthesized depending on the application.

This amphiphilic block copolymer can be crosslinked into a degradable 3-D scaffold for filling bony defects or repairing bone, cartilage, osteochondral, tendon or ligament (such as anterior cruciate ligament) damage. It can also be extruded into fibers to serve as a degradable suture or as a material for fused deposition modeling machines, allowing for the printing of custom scaffolds. The 3-D constructs can also be adapted to deliver therapeutic agents such as growth factors, and stem or progenitor cells (endogenous or exogenous) at the implant site.

Another unique property of HA-PELA composites is their temperature-dependent shape memory behavior. As demonstrated herein, the shape recovery for HA-PELA composites within the physiologically relevant range (e.g., 40-50° C.) is achievable, making these shape memory materials well suited for a variety of medical applications.

For instance, the fabrication of shape memory scaffolds by rapid prototyping enables the manufacturing of customized bone grafts that precisely fit within a tissue defect. It also enables facile graft fixation by compressing the graft into a minimally invasive shape/configuration pre-implantation, and subsequently allowing it to expand post-implantation to precisely conform to the defect. An additional advantage of the HA-PELA scaffolds is that the thermoplastic nature of the un-crosslinked PELA allows the permanent shape be re-programmed at elevated temperatures (e.g., ˜50° C. for 5% HA-PELA). This is not possible with crosslinked thermoset shape memory polymer networks, where the permanent shape is fixed during initial fabrication.

In one aspect, the invention generally relates to a composition comprising a biodegradable amphiphilic block co-polymer. The block co-polymer comprises hydrophilic blocks and degradable hydrophobic blocks and the composition exhibits a shape memory property.

Shape-memory property refers to the ability of a material to return from a deformed state (temporary shape) to its original (permanent) shape induced by an external stimulus (trigger), such as a temperature change.

In certain embodiments, the composition is thermoplastic and at least partially un-crosslinked. In certain embodiments, the composition is thermoplastic and un-crosslinked.

In certain embodiments, the composition exhibits a shape memory property adapted to/characterized by programing and/or reprogramming a permanent shape at an elevated temperature (e.g., from about 30° C. to about 80° C., from about 40° C. to about 70° C., from about 40° C. to about 60° C., from about 40° C. to about 50° C., from about 50° C. to about 80° C., from about 60° C. to about 80° C., 40° C. to about 60° C.).

In certain embodiments, the composition exhibits a shape memory property adapted to/characterized by programing a temporary shape at or below room temperature (e.g., from about 0° C. to about 30° C., 0° C. to about 25° C., 0° C. to about 20° C., 10° C. to about 30° C., 15° C. to about 30° C., 15° C. to about 25° C., 10° C. to about 25° C.).

In certain preferred embodiments, the composition further includes one or more inorganic minerals, for example, selected from the group consisting of calcium apatites, calcium phosphates, hydroxyapatite, and substituted hydroxyapatites.

In certain preferred embodiments, the composition possess a stable structural interface between the co-polymer and the one or more inorganic minerals. In certain preferred embodiments, the one or more inorganic minerals is hydroxyapatite.

The one or more inorganic minerals (e.g., hydroxyapatite) may be present in any suitable percentage depending on the application at hand. In certain embodiments, the one or more inorganic minerals is present in a weight percentage of at least 1% (e.g., at least 5, at least 10%, at least 20%, from about 10% to about 50%, from about 20% to about 50%, from about 20% to about 60%, from about 20% to about 70%, from about 1% to about 70%).

In certain embodiments, the biodegradable amphiphilic block co-polymer includes blocks of poly(ethylene glycol) and polyesters. In certain preferred embodiments, the biodegradable amphiphilic block co-polymer comprises blocks of poly(ethylene glycol) and poly(lactic acid).

In the biodegradable amphiphilic block co-polymer, the hydrophilic block may have any suitable length, for example, from about 1,000 Da to about 100,000 Da (e.g., from about 1,000 Da to about 5,000 Da, from about 5,000 Da to about 10,000 Da, from about 10,000 Da to about 20,000 Da, from about 20,000 Da to about 50,000 Da, from about 50,000 Da to about 100,000 Da).

In certain embodiments, the biodegradable amphiphilic block co-polymer is crosslinked forming a three-dimensional polymer-hydroxyapatite network. In certain embodiments, the biodegradable amphiphilic block co-polymer is characterized by aqueous stability and eletrospinability. In certain embodiments, the composition is a three-dimensional network prepared by rapid prototyping.

In another aspect, the invention generally relates to an article of manufacture made from a composition disclosed herein.

In yet another aspect, the invention generally relates to a biodegradable medical implant. The implant includes a composition comprising a biodegradable amphiphilic block co-polymer, wherein the block co-polymer comprises hydrophilic blocks and degradable hydrophobic blocks, wherein the implant swells and stiffens upon hydration at body temperature.

In certain preferred embodiments, the implant is adapted to self-fixate within a defect upon hydration. In certain embodiments, the implant is a 3-dimensional filler for bony defects, cartilage defects or osteochondral defects. In certain embodiments, the implant is a fibrous membrane wrapped around one or more structural allografts or one or more 3-dimensional synthetic scaffolds to augment tissue repair function. In certain embodiments, the implant is a repair material for bone, cartilage, osteochondral, tendon or ligament damage.

In certain preferred embodiments, the implant is adapted to/capable of supporting attachment of cells (e.g., stem or progenitor cells).

In certain preferred embodiments, the implant is adapted to/capable of supporting attachment of a biological agent (e.g., a growth factor or an antibiotic).

In yet another aspect, the invention generally relates to a biodegradable, three-dimensional composite scaffold, prepared by rapid prototyping from a suspension of hydroxyapatite with an amphiphilic block poly(ethylene glycol-co-lactic acid), wherein the composite scaffold exhibits a shape memory property and swells and stiffens upon hydration at body temperature.

In certain preferred embodiments, the biodegradable, three-dimensional composite scaffold is adapted to/capable of supporting attachment of cells (e.g., stem or progenitor cells). In certain preferred embodiments, the biodegradable, three-dimensional composite scaffold is adapted to/capable of supporting attachment of a biological agent (e.g., a growth factor or an antibiotic). In certain preferred embodiments, the biodegradable, three-dimensional composite scaffold is suitable for implant as a replacement material for bone, cartilage, tendon, ligament, osteochondral damage.

In yet another aspect, the invention generally relates to a method for treating a subject in need of bone or tissue grafting or repair. The method includes: providing a biodegradable medical implant comprising a biodegradable amphiphilic block co-polymer comprising a block co-polymer of hydrophilic blocks and degradable hydrophobic blocks, wherein the implant has attached thereto cells or biological agents; and implanting the biodegradable medical implant in a subject in need thereof to assist bone or tissue grafting or repair.

In certain preferred embodiments, the implant exhibits a shape memory property swells and/or stiffens upon hydration at body temperature.

In certain embodiments, the biodegradable medical implant comprises a three-dimensional composite scaffold prepared from a fibrous composite mesh electrospun from a suspension of hydroxyapatite with an amphiphilic block poly(ethylene glycol-co-lactic acid).

In certain embodiments, the biodegradable medical implant comprises a three-dimensional composite scaffold prepared by crosslinking a suspension of hydroxyapatite with an amphiphilic block poly(ethylene glycol-co-lactic acid).

In certain embodiments, the biodegradable medical implant comprises a three-dimensional composite scaffold prepared by rapid prototyping from a suspension of hydroxyapatite with an amphiphilic block poly(ethylene glycol-co-lactic acid).

The bone or tissue grafting or repair may be any suitable medical procedure, for example, grafting or repair of bone, cartilage, osteochondral, tendon or ligament damage.

### EXAMPLES

### Fabrication and Characterization of PELA and HA-PELA Scaffolds

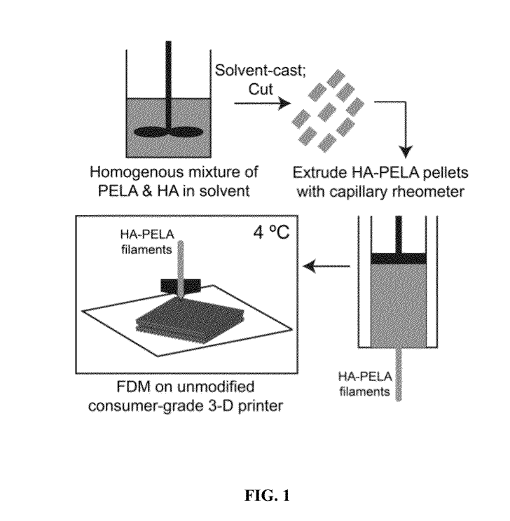

The manufacturing process for 3-D PELA, HA-PELA, and PELA/HA-PELA biphasic scaffolds is depicted in FIG. 1. Briefly, PELA/HA blends were solvent cast into films, extruded into filaments through a capillary rheometer, and then rapid prototyped into 3-D scaffolds by FDM in a sub-ambient printing environment.

The fused deposition modeling (FDM) process consists of feeding a thermoplastic polymer filament through a heated nozzle, guided by software instructions converted from the CAD model, and depositing thin rods of polymer layer by layer that fuse with one another at their contact points. An unmodified consumer-grade 3-D printer, MakerBot® Replicator™ 2X, was used to fabricate the scaffolds. The only “customization” required for printing PELA and HA-PELA polymers were (1) the preparation of PELA and HA-PELA filaments to feed the 3-D printer, and (2) the identification of appropriate environmental and printing nozzle temperatures to support the smooth feeding (without premature softening) and printing (without degradation) of PELA/HA-PELA rather than ABS, the default polymer used for MakerBot® Replicator™ 2X.

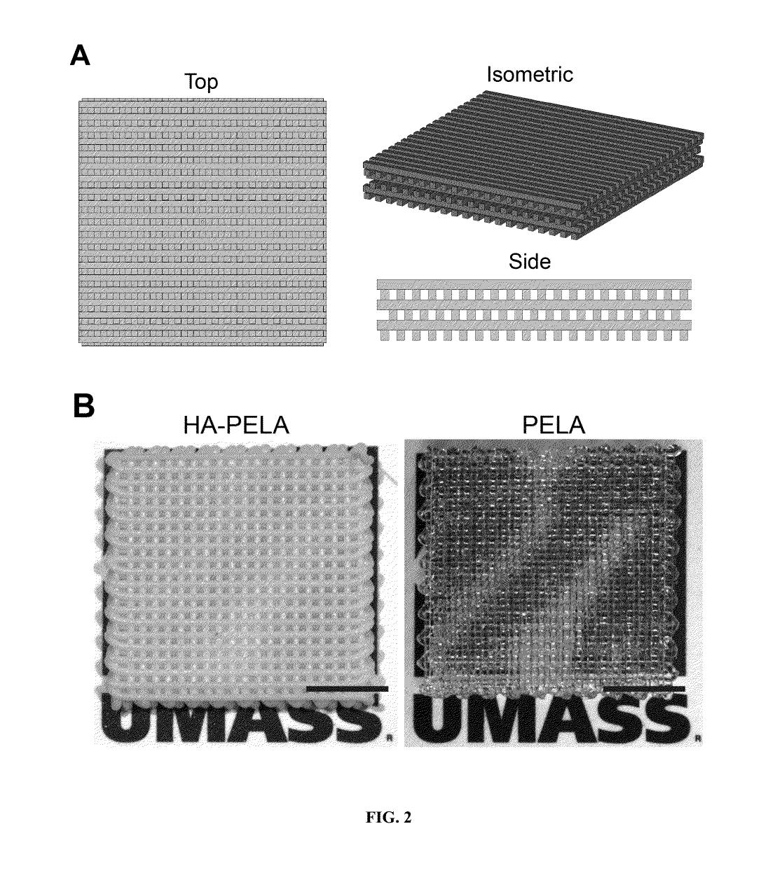

In order to produce the filaments for FDM, a capillary rheometer was used to extrude the PELA and HA-PELA. The capillary rheometer or melt flow indexer allowed for smaller quantities of polymer (gram-scale vs. kilogram-scale required by conventional extruder) to be used. Used for extrusion were pre-fabricated dense films obtained from solvent casting where loose aggregates of HA nanocrystals were homogeneously blended with PELA in the composite. (Song, et al. 2009 J. Biomed. Mater. Res. A 89, 1098-107.) To ensure that the filament was smoothly fed into the heated printing nozzle without premature softening, the FDM was carried out in a deli refrigerator at 4° C., well below the glass transition temperature of PELA (˜19° C.). This temperature prevented the filament from softening/melting and sticking in the drive gear before reaching the printing nozzle, the temperature of which was set at 130° C. for PELA and 160° C. for HA-PELA. This approach allowed the fabrication of PELA and HA-PELA scaffolds with fiber dimensions precisely matching the CAD model (FIG. 2) without undesired line width widening/thinning due to inconsistent extrusion through the heated nozzle. GPC confirmed that the printing nozzle temperature chosen largely maintained the integrity of PELA and HA-PELA composite (Table 1).

TABLE 1
Molecular weight distribution during the processing of PELA
and HA-PELA

| PELA sample | Proc. Temp.^{a} | Mₙ | M_{w} | Mₙ/M_{w} |
|---|---|---|---|---|
| As synthesized PELA | | 85,873 | 134,077 | 1.56 |
| HA-PELA filament | 140° C. | 84,615 | 129,902 | 1.53 |
| HA-PELA scaffold | 160° C. | 82,537 | 130,945 | 1.58 |
| PELA filament | 130° C. | 75,553 | 116,465 | 1.54 |
| PELA scaffold | 130° C. | 76,415 | 117,039 | 1.53 |

^{a}referring to the filament extrusion temperature or the nozzle temperature applied to the prototyping of the scaffolds.

CAD software was used to design 16 mm×16 mm square prism scaffolds with a staggered arrangement of lines (FIG. 2A). The line width and height was set to 0.4 mm, the same as the printing nozzle diameter. The perpendicular and staggered line arrangements between neighboring and alternating layers, respectively, were designed to maximize the retention of cells during initial cell seeding. Line spacing of 0.4 mm, which was shown to be advantageous over large spacing (e.g., 0.8 mm) in achieving sufficient seeding efficiency, was used to give a theoretical scaffold porosity of 61.7%. Six-layer (2.4 mm high) scaffolds were designed for all cell culture studies and 10-layer (4.0 mm high) scaffolds were designed for all physical characterizations. The scaffolds were rapid prototyped based on the CAD models by FDM on an unmodified consumer-grade 3-D printer. Macroscopic images of the scaffolds (FIG. 2B) revealed that their line width was consistent with the CAD model (0.4 mm) Scanning electron micrographs showed a roughened fiber appearance for the HA-PELA composite scaffolds (FIG. 2C) while a smooth fiber appearance for the un-mineralized PELA scaffold (FIG. 2D). Cross-sections of both scaffolds revealed circular fibers with open pores between fibers (FIGS. 2C & 2D).

GPC was used to determine the effect of filament extrusion and rapid prototyping on the molecular weight and polydispersity of PELA (Table 1). PELA underwent a slight decrease in molecular weight, while the molecular weight of HA-PELA was minimally affected by the fiber extrusion at elevated temperatures (130° C. for PELA and 140° C. for HA-PELA). The rapid prototyping of PELA at the same nozzle temperature of 130° C., however, did not lead to further decreases in the molecular weight of the printed PELA scaffold. No significant changes in the molecular weight distributions of PELA and HA-PELA were detected throughout the extrusion and rapid prototyping.

### Swelling Behavior of HA-PELA and PELA

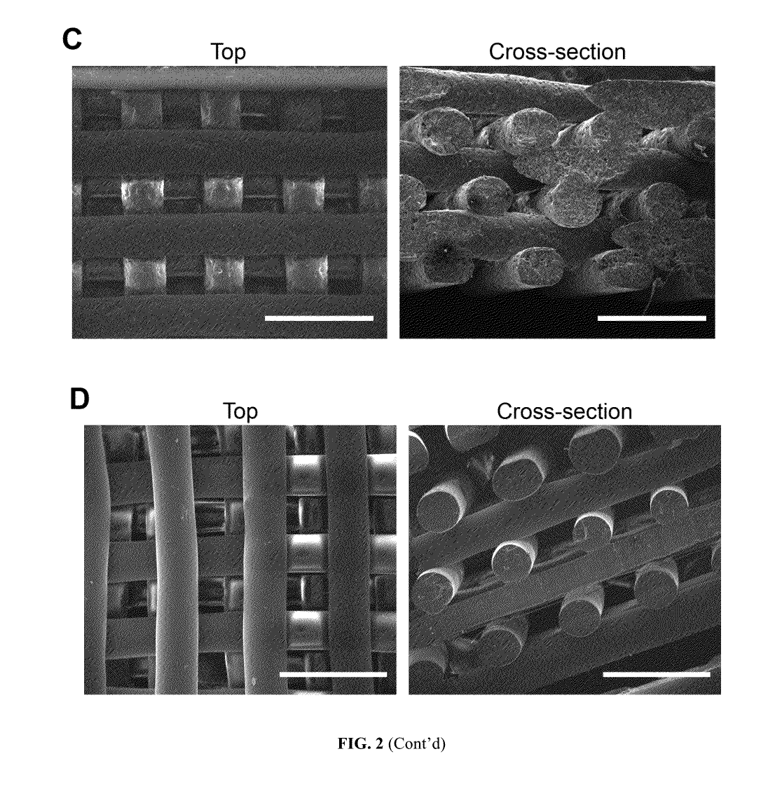

The swelling and water absorption behavior of the HA-PELA and PELA scaffolds (n=3) in deionized water at 37° C. was monitored over time (FIG. 3). The mass and volume of HA-PELA scaffolds increased more rapidly than PELA, resulting in a higher total swelling after 24 h (FIGS. 3A & 3B). The mass and volume of both scaffolds increased more rapidly within the first 2-4 h, followed by slower but continued increases, reaching 75% (mass) and 66% (volume) for HA-PELA, and 34% (mass) and 43.8% (volume) for PELA by 24 h, respectively. The line width of the scaffolds also increased over the 24 h swelling period for both scaffolds (FIG. 3C), with the fully hydrated HA-PELA scaffold exhibiting significantly higher line width than that of PELA.

The incorporation of HA significantly increased the swelling of the scaffolds. This result may be attributed to the further increased hydrophilicity upon HA incorporation and the more roughened HA-PELA fiber morphologies that promoted better water penetration within HA-PELA. These observations support that the 3-D PELA-based scaffolds are highly hydrophilic, in agreement with prior water contact angle and swelling experiments carried out on electrospun PELA meshes and dense solvent-cast PELA films. (Kutikov, et al. 2013 Acta Biomater. 9, 8354-8364; Lee, et al. 2005 Biomaterials 26, 671-8.)

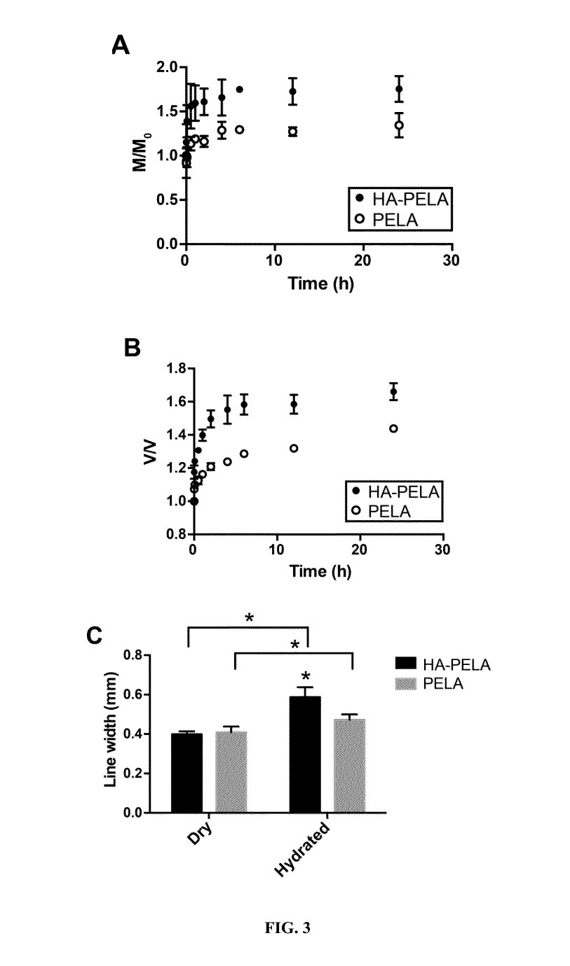

Hydration-induced phase separation of the hydrophilic PEG blocks from the hydrophobic segments was confirmed by modulated differential scanning colorimetry with our electrospun PELA or HA-PELA fibrous meshes and by small-angle x-ray scattering of other related amphiphilic systems. We showed here that the compressive moduli of 3-D HA-PELA were higher than those of the PELA scaffolds in both dry and hydrated states (FIG. 4). The hydration-induced increase in compressive modulus was observed with both scaffolds, but the effect was more pronounced in PELA (˜4-fold increase) than in HA-PELA (1.38-fold increase). However, the fully hydrated HA-PELA scaffold was more than twice as stiff as the hydrated PELA scaffold. The higher (by 6.7-fold) modulus of the dry HA-PELA scaffold compared to the dry PELA supported good structural integration of HA with the amphiphilic polymer. The increase in stiffness of the hydrated HA-PELA suggests that HA did not disrupt the hydration-induced phase separation of PELA.

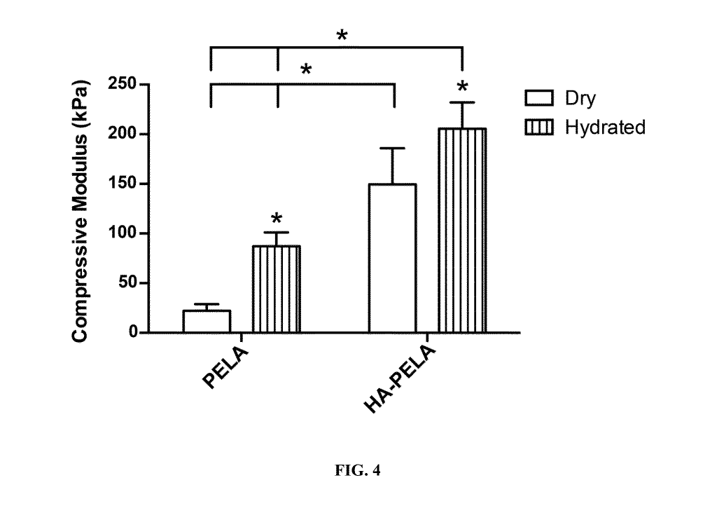

An in vitro pull-out test was developed to quantify the hydration-induced self-fixation behavior. The test specimen was placed into a rapid prototyped testing device (FIG. 5B), allowed to swell in water, and the force required to pull it out of the testing device was measured. While this test does not fully recapitulate the environment of a tissue substrate, it provides a reproducible and facile method to quantitatively compare the self-fixation behavior of various scaffolds in vitro, thereby serving as a valuable, although imperfect, predictor. Significant fixation of both HA-PELA and PELA scaffolds was observed after 2 h of hydration (FIG. 5D). The peak force required to remove the hydrated HA-PELA scaffold was 15-fold higher than that for the dry scaffold. The fixation force measured for the hydrated HA-PELA was also significantly higher than that of hydrated PELA, likely due to a combination of the more pronounced swelling and the more substantial stiffening of the hydrated HA-PELA. This is believed to be the first report of rapid prototyped biomaterial scaffold exhibiting well-characterized hydration-induced self-fixation behavior.

The substantial difference between PELA and HA-PELA in supporting cell adhesion can be exploited for applications where varying degrees of tissue ingrowth are required on opposing sides of a biomaterial scaffold. One such application is guided bone regeneration (GBR). (Retzepi, et al. 2010 Clin. Oral Implants Res. 21, 567-76.) The principle behind GBR is to exclude fibroblasts and soft tissue from occupying the bony defect while encouraging the defect be populated with osteogenic cells and filled with new bone. (Dimitriou, et al. 2012 BMC Med. 10, 81; Dahlin, et al. 1988 Plast. Reconstr. 81, 672-676.)

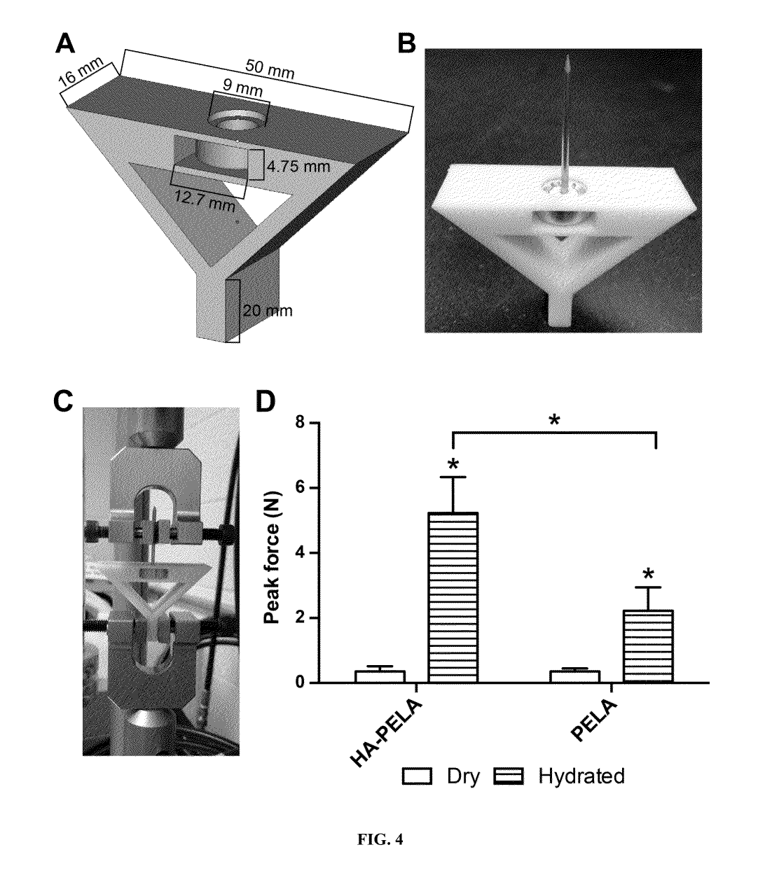

Using a CCK-8 assay, it was shown that the PELA scaffold restricted the adhesion of fibroblasts (FIG. 6A). Only a small number of fibroblasts loosely adhered to the PELA scaffold, as visualized by MTT staining after 24 h (FIG. 6B), and they failed to proliferate over time (FIG. 6B). By contrast, nearly 5 times fibroblasts adhered to the HA-PELA upon cell seeding and they remained viable and stably attached to the scaffold during the 14-day culture period (FIGS. 6A & 6B). The osteoconductive HA-PELA also readily supported the cellular adhesion of MSCs, resulting in 10 times higher seeding efficiency on HA-PELA than on the PELA (FIG. 6C). Combined with previously elucidated highly sensitized response of the MSCs adhered to HA-PELA (as opposed to PELA or HA-PLA) to osteogenic inductions, ¹²these observations establishes 3-D HA-PELA as a promising scaffold for guiding bone regeneration upon surgical implantation to a bony defect. The incorporation of HA effectively offset the low-fouling effect of the PEG component of the amphiphilic composite.

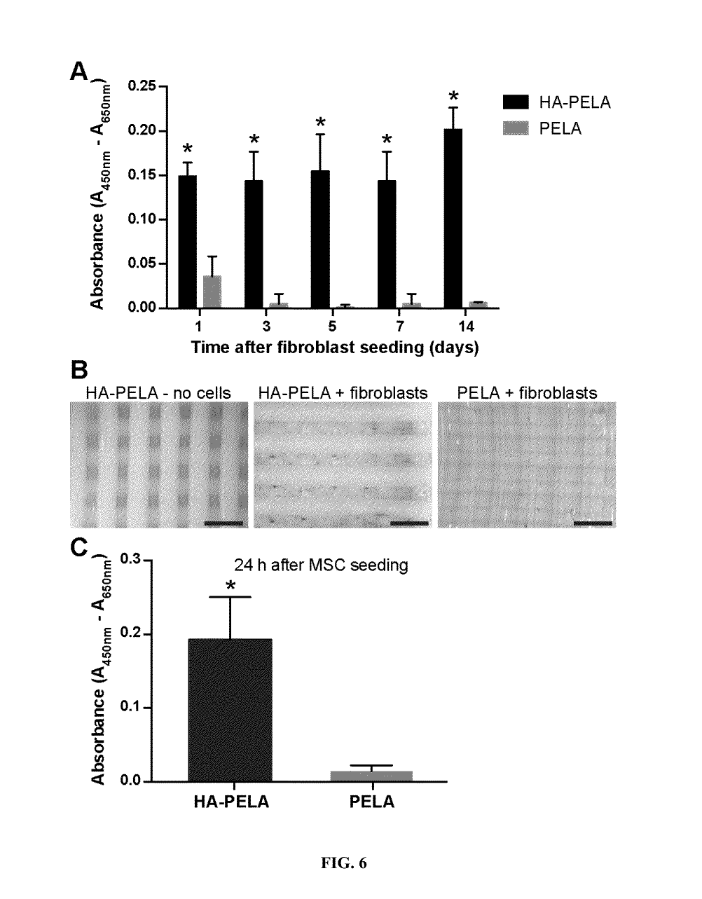

A PELA/HA-PELA biphasic scaffold was fabricated for potential GBR applications using the consumer-grade 3-D printing system (FIG. 6). The top low-fouling PELA phase was designed to prevent fibroblast adhesion/scar tissue encapsulation/soft tissue collapse into the defect in vivo while the bottom HA-PELA phase, upon insertion into a bony defect, was designed to support the attachment of osteoblasts or progenitors residing in the bony tissue environment to encourage bone ingrowth. Outstanding control in line width and clear separation of the distinct phases (FIGS. 7B & 7C) was accomplished in the biphasic construct using the consumer-grade printer.

### Hydration-Induced Stiffening of the Scaffolds

The effect of hydration on the compressive modulus of HA-PELA and PELA scaffolds was determined by unconfined compressive testing at 37° C. In both dry and hydrated states, HA-PELA exhibited a significantly higher compressive modulus than PELA (FIG. 4). After hydration in 37° C. deionized water for 24 h, the compressive moduli of both HA-PELA and PELA significantly increased. The magnitude of hydration-induced stiffening was higher for PELA than HA-PELA, with an increase in compressive modulus of 395% compared to 37.5%.

### Hydration-Induced Self-Fixation of Scaffolds in a Simulated Confined Defect

A device was designed to assess how the hydration-induced swelling and stiffening of the HA-PELA and PELA scaffolds may be exploited to facilitate their stable self-fixation into skeletal tissue defects as synthetic bone grafts. The CAD model and rapid prototyped ABS holder (FIG. 5A) incorporated a cylindrical aluminum spacer to hold a cylindrical test specimen with a bottom stem and a drywall nail penetrating through the center axis of the specimen (FIG. 5B) to fit in the grips of a MTS mechanical testing system. This design allows convenient placement of a test specimen in a precisely configured confined cylinder to allow for a pull-out test to be reproducibly carried out on any standard mechanical testing machine (FIG. 5C). The peak force required to pull the scaffold out of the specimen holder via the nail grip was determined. This force was measured for dry HA-PELA and PELA scaffolds and for scaffolds pre-swelled in the fixation device in deionized water at 37° C. for 2 h. The peak force increased by 15-fold and 6.3-fold following hydration of HA-PELA and PELA scaffolds in the fixation device, respectively (FIG. 5D). The peak fixation force of the hydrated HA-PELA scaffolds was significantly higher than that of the hydrated PELA scaffolds. The observed increase in peak force upon hydration, positively correlated with the difficulty of pulling out the specimen, is a potential indicator of how the specimens may swell/stiffen and become stably fixated within a tissue defect.

### NIH3T3 Attachment and Proliferation on the Rapid Prototyped Scaffolds

A CCK-8 assay was used to quantify the viability of NIH3T3 fibroblasts cultured on HA-PELA, PELA, and biphasic scaffolds (FIG. 6A). The CCK-8 reagent has low toxicity and the colored formazan product is soluble in media, allowing the cellular viability on the same scaffolds to be longitudinally monitored in a non-destructive manner. Initial cell attachment was significantly higher on HA-PELA than PELA or the biphasic scaffolds, and the much higher cellular viability was maintained on HA-PELA for 14 days. The extremely poor cellular attachment on PELA resulted in further cell death, leaving few viable cells on PELA by day 3. Differences in cell attachment between HA-PELA and PELA were further confirmed by staining viable cells with formazan dye (FIG. 6B). The HA-PELA scaffolds supported the attachment of viable cells evenly distributed across different layers of the composite scaffold. The PELA scaffold, however, only contained a small number of viable cells trapped within the pores, with few cells directly adhered to the low-fouling fibers.

### MSC Attachment on the Rapid Prototyped Scaffolds

The MSC attachment on HA-PELA was assessed in order to determine the suitability of HA-PELA for supporting potential stem/progenitor cell attachment and bone in-growth in vivo. CCK-8 assay revealed significantly higher (e.g., 16-fold increase in viable cells at 24 h) seeding efficiency of MSCs on the 3-D HA-PELA scaffolds than on the PELA scaffolds (FIG. 6C), further supporting the cell-adhesive nature/osteoconductivity of the former and the low-fouling nature of the latter.

### Rapid Prototyping Biphasic PELA/HA-PELA Scaffolds

Biphasic scaffolds composed of 3 PELA layers and 3 HA-PELA layers were designed using CAD (FIG. 7A) and fabricated by FDM. Stereomicroscopy images showed distinct yet well-connected PELA and HA-PELA phases (FIG. 7B). Scanning electron microscopy and EDX mapping of the cross-section of the biphasic scaffold confirmed the distinct mineral composition in the HA-PELA fibers (FIG. 7C). The calcium (Ca) and phosphate (P) signals were clearly localized within and on the surface of the HA-PELA fibers while only minor background noise was detected in the adjacent PELA phase.

### Shape Memory Performance of HA-PELA Composites

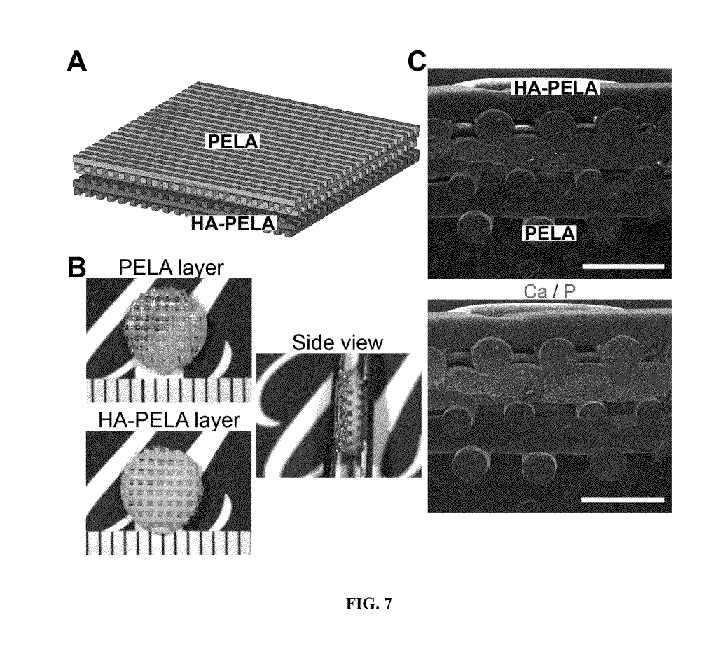

HA-PELA composites exhibit temperature-dependent shape memory behavior. Dynamic mechanical testing indicates that the storage modulus of HA-PELA with 0-50 wt % HA sharply drops as temperature increases (FIG. 8). This indicates that the shape recovery for HA-PELA composites within the physiologically relevant range (e.g., 40-50° C.) is feasible, making these shape memory materials well suited for medical applications.

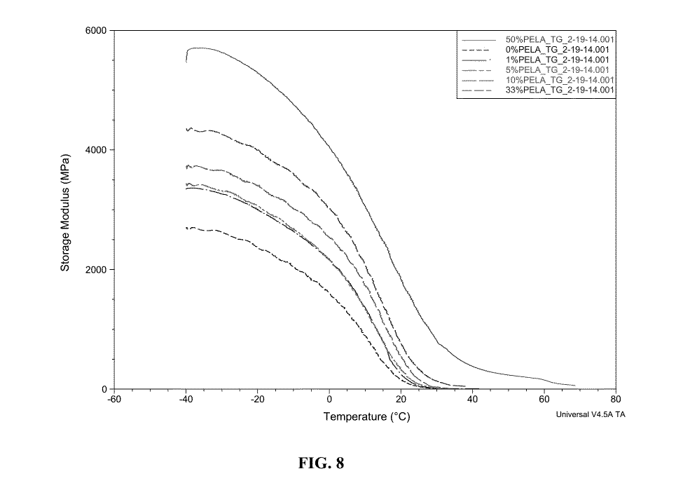

The thermoplastic nature of PELA allows the fabrication of shape memory scaffolds by rapid prototyping, as demonstrated in FIG. 9. It enables the manufacturing of customized bone grafts that precisely fit within a tissue defect. It also enables facile graft fixation by compressing the graft into a minimally invasive shape/configuration pre-implantation, and subsequently allowing it to expand post-implantation to precisely conform to the defect. As a proof of concept, a rapid prototyped 25% HA-PELA scaffold was compressed into a temporary shape at room temperature, and then allowed to recover into the original rapid prototyped shape upon submerging the scaffold in 50° C. water bath (FIG. 8).

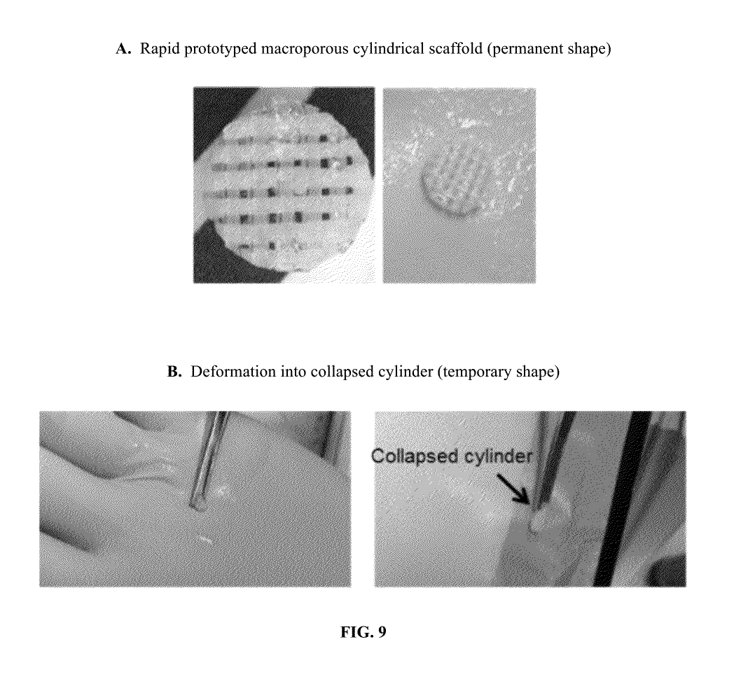

An additional advantage of the HA-PELA scaffolds is that the thermoplastic nature of the un-crosslinked PELA allows the permanent shape be re-programmed at elevated temperatures (e.g., ˜50° C. for 5% HA-PELA). This is not possible with crosslinked thermoset shape memory polymer networks, where the permanent shape is fixed during initial fabrication. As a proof of concept, a solvent cast film of HA-PELA with an original permanent shape of a straight bar (from the casting mold) was deformed at room temperature into a temporary spiral shape (FIG. 10A). When submerged into a 50° C. water bath, it instantly (in about 2 seconds) recovered into the original straight bar (FIG. 10A). Subsequently, the composite was reprogrammed into a spiral configuration by deforming the bar of HA-PELA into a spiral while submerged in water at 50° C. (FIG. 10B). This HA-PELA spiral can subsequently deformed into a temporary flat bar shape at room temperature. Upon submerging the HA-PELA bar in 50° C. water bath, it instantly recovered into the reprogrammed permanent spiral shape (FIG. 10B).

### In Vivo Application of Electrospun HA-PELA Composites

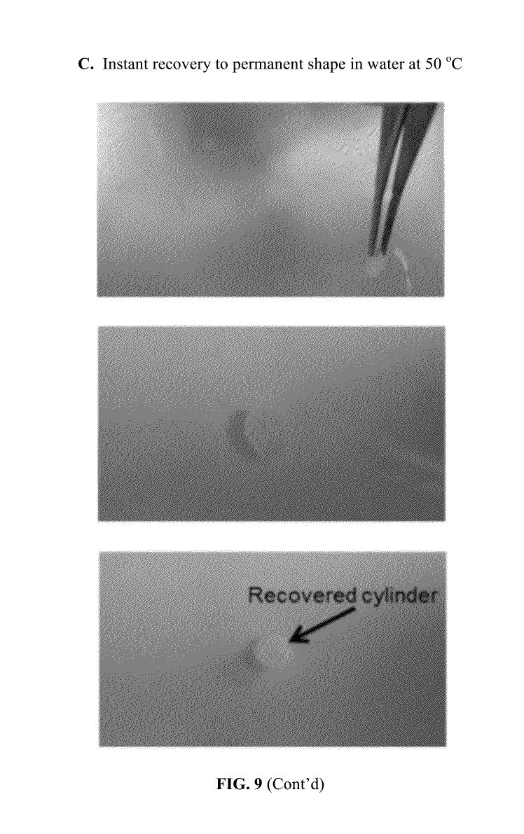

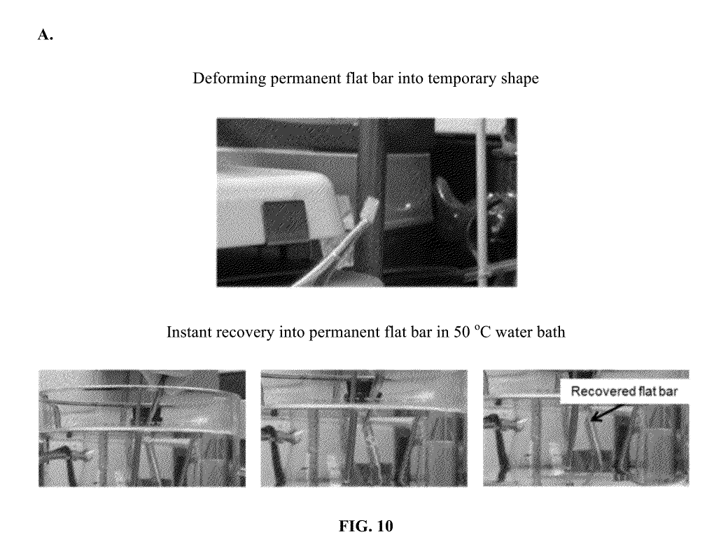

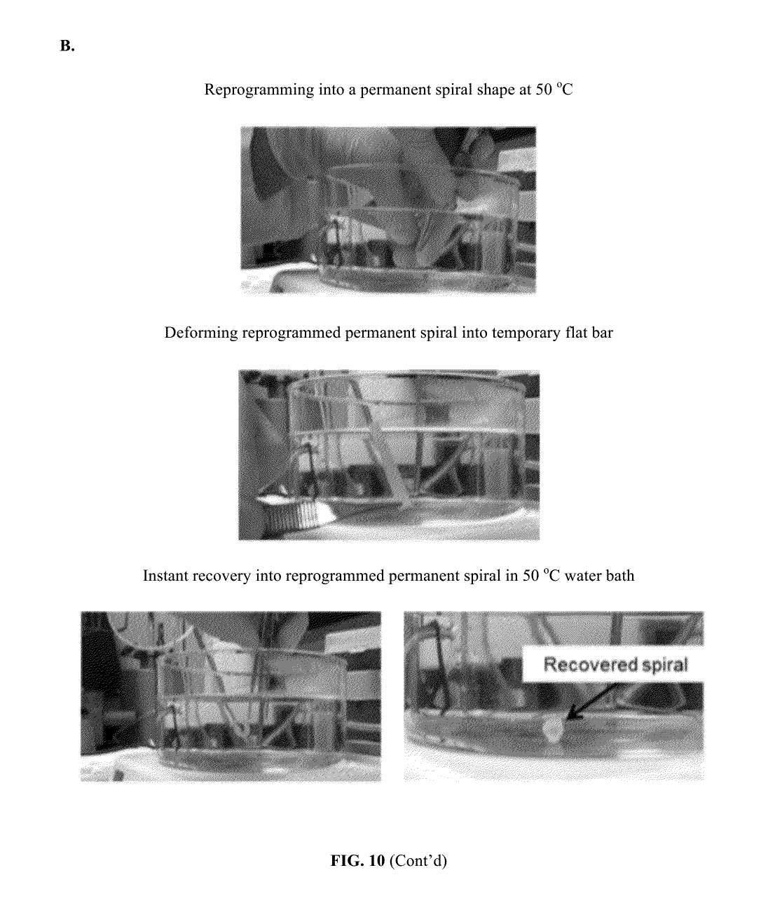

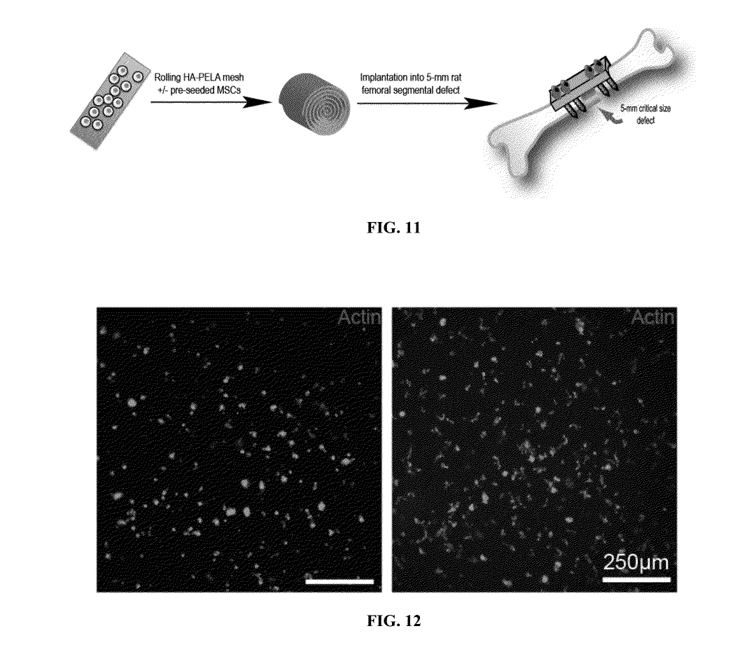

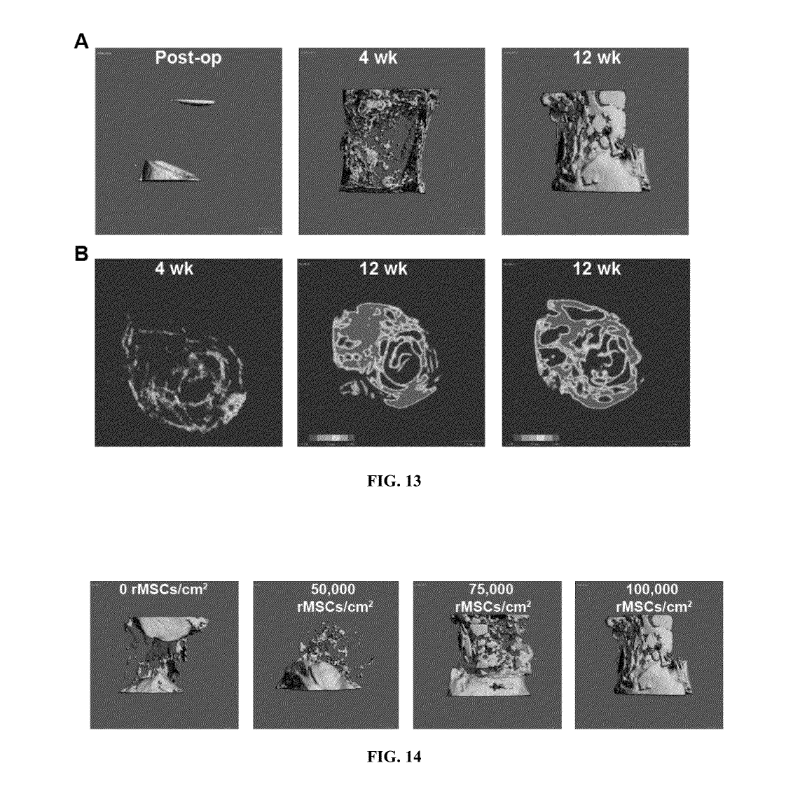

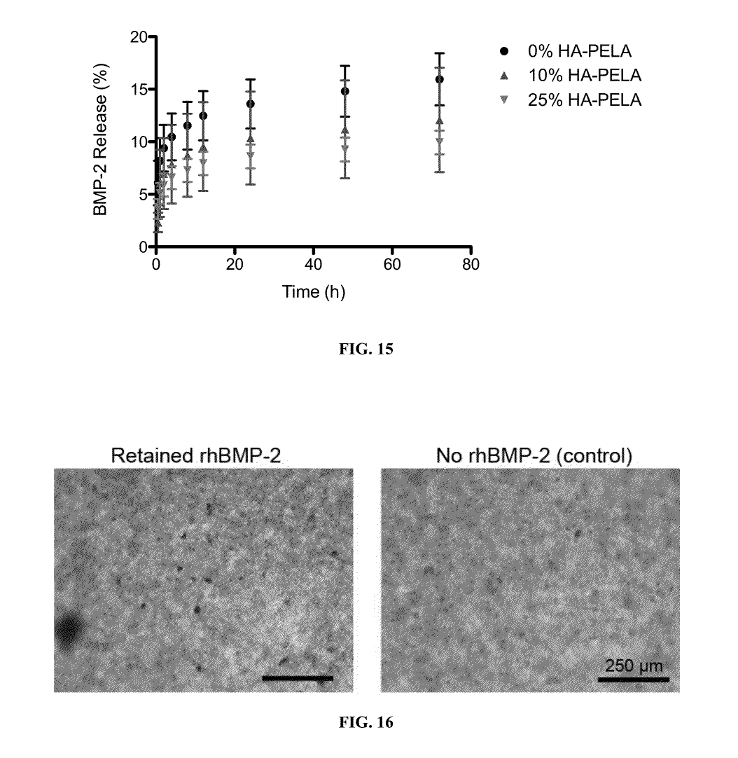

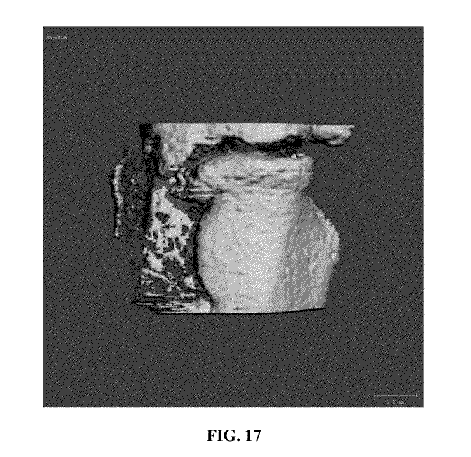

Electrospun HA-PELA scaffolds, with or without pre-seeded rMSCs or 500 ng rhBMP-2, were manually wrapped into cylindrical spirals and implanted into 5-mm rat femoral defects (FIG. 11). rMSC attachment onto the scaffolds, containing either 10% or 25% HA by weight, was confirmed by F-actin staining (FIG. 12A & B). The addition of exogenous rMSCs was shown to promote templated bone formation over a 12-week period (FIG. 13). The amount of templated bone formed increased with rMSC number (FIG. 14), supporting the role of rMSCs. For an alternative or complementary treatment strategy, a low dose (500 ng) of rhBMP-2 was loaded into the electrospun HA-PELA. The addition of HA improved the retention of rhBMP-2 (FIG. 15), though the nanofibrous nature of PELA alone also facilitated rhBMP-2 binding and release. Even after 7 days, the rhBMP-2 remained bioactive, as evidenced by the transdifferentiation of C2C12 myoblasts adhered to the scaffolds (FIG. 16). Furthermore, the low dose of rhBMP-2 was sufficient to induce robust bone formation in vivo by 4 weeks post-op (FIG. 17).

### Experimental

### Materials

3,6-Dimethyl-1,4-dioxane-2,5-dione (D,L-lactide) was purchased from Sigma-Aldrich (St. Louis, Mo.), purified by recrystallization twice in anhydrous toluene, and dried under vacuum prior to use. Poly(ethylene glycol) (20,000 Dalton, BioUltra) was purchased from Fluka (Switzerland). Polycrystalline hydroxyapatite powder (consisting of loose aggregates of ˜100-nm crystallites) was purchased from Alfa Aesar (Ward Hill, Mass.). All other solvents and reagents were purchased from Sigma-Aldrich (St. Louis, Mo.) and used as received.

### Polymer Synthesis

Poly(D,L-lactic acid)-poly(ethylene glycol)-poly(D,L-lactic acid) (PELA) tri-block copolymer was synthesized and characterized as previously described. ¹²Briefly, melt ring opening polymerization of D,L-lactide (0.12 mol) was initiated by poly(ethylene glycol) (20,000 Dalton, 0.2 mmol) with Tin(II) 2-ethylhexanoate (˜95%, 0.06 mmol) catalysis. The reaction proceeded at 130° C. for 5 hours under argon. The crude PELA was dissolved in chloroform, purified by precipitation in methanol, and dried under vacuum before being subjected to GPC characterizations.

### Preparation of PELA and HA-PELA Films

PELA and HA-PELA dense films (˜1.6 mm thick) were produced by solvent casting and sectioned into —0.5×0.5 cm² pellets for filament extrusion. For the fabrication of HA-PELA composite films, HA (3.3 g, 25% w/w PELA) was bath-sonicated in 20 mL chloroform for 30 min. PELA (10 g) was added and the mixture was stirred overnight. The HA-PELA mixture was subsequently poured into Teflon molds. The chloroform was evaporated in a fume hood at room temperature overnight and subsequently in a vacuum oven at 60° C. for 24 h. PELA films were prepared by evaporating a chloroform solution of PELA without HA in the same mold followed by vacuum drying under identical conditions.

### Filament Extrusion

The PELA and HA-PELA filaments were extruded using a LCR7000 capillary rheometer (Dynisco Instruments, Franklin, Mass.) through a 2.81-mm diameter die. The barrel was preheated at 130° C. (for PELA) or 140° C. (for HA-PELA) for ˜90 sec before the PELA or HA-PELA pellets were loaded, followed by continued heating at the respective temperatures for 120 sec. The filaments were extruded through the die with a 120-sec run time and a barrel piston speed of 32.84-mm/min and collected manually.

### 3-D Scaffold Fabrication

A 3-D CAD model of a 16 mm×16 mm square prism (FIG. 1a, 2.4 mm or 4 mm in height) was designed in 3-Matics (Materialise, Belgium) and converted into g-code instructions by MakerWare (MakerBot Industries, Brooklyn, N.Y.). A MakerBot® Replicator™ 2X 3-D printer (MakerBot Industries, Brooklyn, N.Y.) cooled in a deli refrigerator at 4° C. was used to print the scaffolds using the PELA or HA-PELA filaments. The sub-ambient printing environment was required to cool PELA below its T_{g} (˜19° C.) so that the filament could be continuously fed into the printer without undesired softening before reaching the heated printing nozzle. Nozzle temperatures of 130° C. and 160° C. were applied to print the PELA and HA-PELA, respectively. The build platform was maintained at 30° C. to ensure stable adhesion of the bottom printed layer to the platform. Scaffolds were printed with a platform feed rate of 90 mm/sec.

Biphasic PELA/HA-PELA scaffolds were fabricated by extruding 3 layers of HA-PELA followed by 3 layers of PELA. PELA and HA-PELA filaments were loaded into separate nozzles of the Replicator™ 2X. The same printing conditions described above for printing PELA and HA-PELA were applied accordingly.

### Gel Permeation Chromatography (GPC)

PELA and HA-PELA composites were dissolved in THF, centrifuged (720×g, 5 min) to pellet the HA, before the supernatant was collected and filtered with a 0.4-μm Teflon filter for GPC analyses. Molecular weights and polydispersity of PELA was determined by gel permeation chromatography (GPC) on a Varian Prostar HPLC system equipped with two 5-mm PLGe1 MiniMIX-D columns (Agilent, Santa Clara, Calif.) and a PL-ELS2100 evaporative light scattering detector (Polymer Laboratories, UK). THF was used as an eluent at 0.3 mL/h at room temperature. Molecular weight and polydispersity calculations were calibrated with EasiVial polystyrene standards (Agilent, Santa Clara, Calif.).

### Optical Imaging

Macroscopic optical images of the HA-PELA, PELA, and the biphasic scaffolds were taken on a Leica M50 stereomicroscope equipped with a Leica DFC295 digital camera (Leica Microsystems, Germany).

### Scanning Electron Microscopy and Associated Energy-Dispersive X-Ray Spectroscopy (EDX)

HA-PELA, PELA, and PELA/HA-PELA biphasic scaffolds were coated with 3 nm of carbon and imaged on a Quanta 200 FEG MKII scanning electron microscope (FEI Inc., Hillsboro, Oreg.) under high vacuum at 10 kV. EDX was carried out to map the elemental compositions (Ca and P) of the biphasic scaffold at 15 kV with an Oxford-Link INCA 350 x-ray spectrometer (Oxford Instruments, United Kingdom).

### Porosity Calculation

The theoretical porosity (P) of the scaffolds was calculated by determining the percentage (%) of scaffold volume that is occupied by the polymer rods, as described by Zein et al. and shown in equation (1):

$$P = \frac{Va - Vt}{Va} \times 100 \% \qquad ( 1 )$$

where Va (mm³) is the apparent scaffold volume and Vt is the scaffold true volume taken up by polymer. (Zein, et al. 2002 Biomaterials 23, 1169-85.) Assuming that the FDM polymer rods are cylindrical in shape with a uniform diameter, the true volume taken up by polymer (Vt) in a square prism can be calculated as

Vt=L×N×Vrw (2)

where L is the number of rods per layer, N is the number of layers, and Vrw is the volume of each cylindrical rod which is determined by the printed line width and length.

### Swelling Behavior

The height and diameter (averaged from 3 measurements) of dry PELA and HA-PELA scaffolds (n=3), cored from the square prism FDM blocks using a biopsy punch, was measured with a digital caliper. Line width was averaged from 5 measured lines per scaffold using a light microscope (Axioscop 2 MAT; Carl Zeiss, Germany) and ImageJ (National Institutes of Health, Bethesda, Md.). Scaffold mass was weighed using an analytical balance (ML104; Mettler-Toledo, Columbus, Ohio). Hydrated scaffold dimensions and mass were measured at various time intervals following incubation in de-ionized water at 37° C. Residual water was removed prior to weighing by briefly blotting the scaffolds on KimWipes. Change in mass (M/M₀) was calculated by dividing the mass following water equilibration (M) by the initial mass of a scaffold briefly submerged in water (M₀). Change in volume (V/V₀) was calculated in the same manner. Hydrated line width was measured following 24-h incubation in 37° C. deionized water.

### Mechanical Testing

The compressive modulus of PELA and HA-PELA scaffolds (n=3) was determined on a Q800 DMA equipped with a liquid nitrogen gas cooling accessory (TA Instruments, New Castle, Del.). Cylindrical specimens 6 mm in diameter and 4 mm in height, the dimensions used by Moroni et al. for characterizing mechanical properties of macroporous scaffolds, ¹⁹were cored from the square prism FDM blocks. Unconfined compressive testing (N=3) was performed at 37° C. for both dry (as-printed) and hydrated (24 h in deionized water) scaffolds. The height and diameter of each specimen was measured with a digital caliper prior to testing. Each specimen was held isothermal at 37° C. for 30 min before being pre-loaded with a force of 0.001N and ramped at a rate of 1.0 N/min to 10 N. The compressive modulus was recorded as the slope of the linear region (0 to 0.5% strain) of the stress/strain curve.

### Pull-Out Test

A custom sample holder (FIG. 4A) simulating a confined circular tissue defect was developed to enable quantitative measurement of the hydration-induced swelling/stiffening effect of the scaffolds via a pull-out test. A CAD model of the sample holder was designed in 3-Matics and fabricated on a MakerBot Thing-O-Matic™ 3-D printer using acrylonitrile butadiene styrene (ABS). In order to ensure consistent specimen placement, the specimen holder portion of the ABS prototype was tight-fitted with a standard cylindrical aluminum spacer (12.7 mm OD×6.35 mm ID×4.76 mm H, W.W. Grainger Inc., Chicago, Ill.). Cylindrical PELA or HA-PELA scaffolds 6 mm in diameter and 4 mm in height, cored from square prism FDM blocks using a biopsy punch, were each drilled with a center axial hole 1.6 mm in diameter to enable the insertion of a drywall nail (1.6 mm diameter, 32 mm long, 3.8 mm diameter head, FIG. 4B). The specimen was inserted into the aluminum spacer, and either tested dry or equilibrated in deionized water within the holder for 2 h at 37° C. prior to test. The bottom stem of the custom ABS holder and the sharp end of the inserted nail were secured between the grips of a MTS Bionix 370 mechanical testing system (MTS Systems Corporation, Minneapolis, Minn.), respectively (FIG. 4C). Specimens were ramped at a rate of 50 mm/min until they are completely pulled out of the ABS/aluminum holder to determine the peak force as recorded by a 250N load cell (Interface, Scottsdale, Ariz.).

### Cell Attachment and Proliferation

HA-PELA and PELA scaffolds (6.3 mm in diameter, 2.4 mm in height) were washed 3 times in deionized water (5 min per wash), sterilized in 70% ethanol, and allowed to air dry in a laminar flow hood. Residual ethanol was removed with a wash in PBS followed by equilibration overnight in Dulbecco's Modified Eagle Medium (DMEM, high glucose; Life Technologies, Grand Island, N.Y.) supplemented with 10% bovine calf serum and 1% penicillin/streptomycin. Immediately prior to cell seeding, media were removed from the scaffolds by vacuum and the scaffolds were transferred to ultra low-attachment 24-well plates (Corning Inc., Corning, N.Y.). NIH3T3 fibroblasts were trypsinized from adherent culture and seeded on the scaffolds (200,000 cells in 50 μl of media), and allowed to attach in an incubator (37° C., 5% CO₂) for 1 h.

Bone marrow-derived stromal cells (MSCs) were isolated from 289-300 g male Charles River SD rats according to the procedure approved by the University of Massachusetts Medical School Institutional Animal Care and Use Committee, and enriched by adherent culture as previously described. (Song, et al. 2009 J. Biomed. Mater. Res. A 89, 1098-107.) The cells were cultured in aMEM (without ascorbic acid) containing 20% FBS, 1% penicillin-streptomycin and 2% L-glutamine. Passage 3 MSCs were seeded onto the scaffolds (200,00 cells in 50 μl of media), and allowed to attach in an incubator (37° C., 5% CO₂) for 1 h.

A Cell Counting Kit-8 assay (CCK-8; Dojindo Molecular Technologies Inc., Japan) was performed to assess the viability of cells attached on the scaffolds. At each time point, cell-laden scaffolds were transferred to a fresh well containing 0.7 mL of media and 9% (v/v) CCK-8 reagent. After 4-h incubation, 100 μL of media was removed for measurement of absorbance at 450 nm with 650 nm background correction on a Multiskan FC microplate photometer (Thermo Scientific, Billerica, Mass.). The remainder of the media was aspirated, the scaffolds were washed with PBS, and replaced with fresh media for continued culture up to 14 days. The CCK-8 assay was carried out at day 1, 3, 5, 7 and 14.

NIH3T3 attachment on the HA-PELA and PELA scaffolds was also visualized by staining the viable cells with formazan dye using a MTT kit (Cell Proliferation Kit I; Roche, Indianapolis, Ind.). At 24 h post seeding, scaffolds were transferred to a fresh well containing media with 9% (v/v) MTT labeling reagent. After 3 h of incubation, the scaffolds were imaged on a Leica M50 stereomicroscope equipped with a Leica DFC295 digital camera (Leica Microsystems, Germany).

### Statistical Analysis

All data are presented as mean±standard deviation. Statistical analysis was performed using ANOVA with Tukey post-hoc.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### Equivalents

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A composition comprising a biodegradable amphiphilic block co-polymer, wherein the block co-polymer comprises hydrophilic blocks and degradable hydrophobic blocks, wherein the composition exhibits a shape memory property.

2. The composition of claim 1, wherein the composition is un-crosslinked.

3. The composition of claim 1, wherein the shape memory property is adapted to programing and/or reprogramming a permanent shape at an elevated temperature from about 30° C. to about 80° C.

4. (canceled)

5. (canceled)

6. The composition of claim 1, wherein the shape memory property is adapted to programing a temporary shape at or below room temperature from about 10° C. to about 30° C.

7. (canceled)

8. (canceled)

9. The composition of claim 1, further comprising one or more inorganic minerals selected from the group consisting of calcium apatites, calcium phosphates, hydroxyapatite, and substituted hydroxyapatites.

10. (canceled)

11. (canceled)

12. The composition of claim 9, wherein the composition possess a stable structural interface between the co-polymer and the one or more inorganic minerals.

13-17. (canceled)

18. The composition of claim 1, wherein the biodegradable amphiphilic block co-polymer comprises blocks of poly(ethylene glycol) and polyesters.

19. The composition of claim 1, wherein the biodegradable amphiphilic block co-polymer comprises blocks of poly(ethylene glycol) and poly(lactic acid).

20. The composition of claim 1, characterized by aqueous stability and eletrospinability.

21. The composition of claim 1, wherein the biodegradable amphiphilic block co-polymer is crosslinked forming a three-dimensional polymer-hydroxyapatite network.

22. The composition of claim 1, wherein the composition is a three-dimensional network prepared by rapid prototyping.

23. An article of manufacture made from a composition of claim 1.

24. A biodegradable medical implant comprising a composition of claim 1, wherein the implant swells and stiffens upon hydration at body temperature.

25. The biodegradable medical implant of claim 24, wherein the implant is adapted to self-fixate within a defect upon hydration.

26. The biodegradable medical implant of claim 24, wherein the implant is a 3-dimensional filler or a repair material for for bony defects, tendon or ligament damages, cartilage defects or osteochondral defects.

27. The biodegradable medical implant of claim 24, wherein the implant is a fibrous membrane wrapped around one or more structural allografts or one or more 3-dimensional synthetic scaffolds to augment tissue repair function.

28. (canceled)

29. The biodegradable medical implant of claim 24, wherein the implant is adapted to supporting attachment of cells or a biological agent.

30-32. (canceled)

33. A biodegradable, three-dimensional composite scaffold, prepared by rapid prototyping from a suspension of hydroxyapatite with an amphiphilic block poly(ethylene glycol-co-lactic acid), wherein the composite scaffold exhibits a shape memory property and swells and stiffens upon hydration at body temperature.

34-36. (canceled)

37. A method for treating a subject in need of bone or tissue grafting or repair, comprising:
providing a biodegradable medical implant comprising a biodegradable amphiphilic block co-polymer comprising a block co-polymer of hydrophilic blocks and degradable hydrophobic blocks, wherein the implant has attached thereto cells or biological agents; and
implanting the biodegradable medical implant in a subject in need thereof to assist bone or tissue grafting or repair.

38-42. (canceled)
